SEQUENCE LISTING (10 sequences)

SEQ ID NO: 1 - protein, 135 aa, Artificial Sequence
  RLYB212 Heavy Chain Variable (VH) Region
QVQLQQSGPGLVKPSQTLSLTCAISGDSVSSNSAAWNWIRQSPSRGLEWLGRTYFRSNWYNDYAASVKSRITINQDTSKNQLSLQLNSVTPEDTAVYYCARDGAWGGSSWWPGLPHHYYSGMDVWGQGTTVTVSS

SEQ ID NO: 2 - protein, 108 aa, Artificial Sequence
  RLYB212 Light Chain Variable (VL) Region
EIVLTQSPATLSLSPGERATLSCRASQSVSSYLAWYQQKPGQAPRLLIYDASKRATGIPARFSGSGSGTDFSLTIRSLEPEDFAVYYCQQRSDWQGLTFGGGTKVEIK

SEQ ID NO: 3 - protein, 10 aa, Artificial Sequence
  RLYB212 VH Complementarity Determining Region (CDR) 1
GDSVSSNSAA

SEQ ID NO: 4 - protein, 9 aa, Artificial Sequence
  RLYB212 VH CDR 2
TYFRSNWYN

SEQ ID NO: 5 - protein, 25 aa, Artificial Sequence
  RLYB212 VH CDR 3
ARDGAWGGSSWWPGLPHHYYSGMDV

SEQ ID NO: 6 - protein, 6 aa, Artificial Sequence
  RLYB212 VL CDR 1
QSVSSY

SEQ ID NO: 7 - skipped

SEQ ID NO: 8 - protein, 10 aa, Artificial Sequence
  RLYB212 VL CDR 3
QQRSDWQGLT

SEQ ID NO: 9 - protein, 465 aa, Artificial Sequence
  RLYB212 Heavy Chain
QVQLQQSGPGLVKPSQTLSLTCAISGDSVSSNSAAWNWIRQSPSRGLEWLGRTYFRSNWYNDYAASVKSRITINQDTSKNQLSLQLNSVTPEDTAVYYCARDGAWGGSSWWPGLPHHYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  RLYB212 Light Chain
EIVLTQSPATLSLSPGERATLSCRASQSVSSYLAWYQQKPGQAPRLLIYDASKRATGIPARFSGSGSGTDFSLTIRSLEPEDFAVYYCQQRSDWQGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0141067 A1
(43) Pub. Date: May 2, 2024

(54) Administration of Anti-HPA-1a Antibodies

(71) Applicant: Rallybio IPA, LLC, New Haven, CT (US)

(72) Inventors: Steven Ryder, Westport, CT (US); Douglas L. Sheridan, Branford, CT (US)

(21) Appl. No.: 18/575,805

(22) PCT Filed: Jun. 30, 2022
(86) PCT No.: PCT/US2022/035679

§ 371 Date: Dec. 29, 2023

Related U.S. Application Data
(60) Provisional application No. 63/217,637, filed on Jul. 1, 2021.

(51) Int. Cl. C07K 16/34; A61P 37/06

(52) U.S. Cl. CPC C07K 16/34; A61P 37/06; A61K 2039/505

(57) ABSTRACT

Provided is a regimen for administration of anti-HPA-1a antibodies to a pregnant subject for prevention of maternal alloimmunization with HPA-1a and fetal and neonatal alloimmune thrombocytopenia (FNAIT).

### CROSS-REFERENCE TO RELATED APPLICATIONS

This application claims the benefit of priority of U.S. Provisional Patent Application No. 63/217,637, filed on Jul. 1, 2021.

### BACKGROUND

There are two predominant forms of human platelet antigen 1 (HPA-1) expressed on the surface of platelets: HPA-1a and HPA-1b. Fetal and neonatal alloimmune thrombocytopenia (FNAIT) is a disorder caused by a mismatch in the type of HPA-1 that is expressed by an expectant mother and her fetus. The incompatibility of HPA-1 is due to a Leu/Pro polymorphism at residue 33 of integrin β3 glycoprotein (GP IIIa), which is present in the platelet membrane in complex with integrin αII glycoprotein (GP IIb) to function as a receptor for fibrinogen (Newman et al., 1989). If the woman is negative for the HPA-1a antigen (HPA-1b homozygous), fetal HPA-1a positive platelets that have been inherited from the father (HPA-1a homozygous or heterozygous) and that enter the maternal circulation can induce production of maternal anti-HPA-1a antibodies in a process known as alloimmunization (Göhner et al., 2017).

There are no currently available treatments for the prevention of alloimmunization of an HPA-1a-negative pregnant woman with an incompatible (HPA-1a-positive) fetus. Because there is no prophylactic treatment, pregnant women are not screened for HPA-1 incompatibility, and those who are at risk for FNAIT occurrence are generally not identified until after they have had a child born with confirmed or suspected FNAIT (ACOG Practice Bulletin No. 207, 2019). In subsequent pregnancies, the current recommended therapy for at-risk women is human intravenous immune globulin (IVIG), beginning at Gestational Week 12 and continued throughout pregnancy (Pacheco et al., 2011). Importantly, IVIG does not prevent the occurrence of maternal alloimmunization, does not eliminate the risk of FNAIT occurrence in a subsequent pregnancy with maternal-fetal incompatibility and, in the doses administered, is accompanied by reports of poor tolerability (Vitiello et al., 2019).

Babies with FNAIT are typically diagnosed at the time of delivery by the presence of low platelet counts, the presence of petechiae on the skin, or the manifestations of severe complications such as intracranial hemorrhage or gastrointestinal bleeding. In the neonate, platelet transfusion is the first line therapy for thrombocytopenia, although studies are too small to confirm whether the transfusions are effective at reducing neonatal morbidity or mortality (Lieberman et al., 2019). The aim of the transfusion is to maintain an acceptable platelet level within the first 72 to 96 hours of life (Espinoza et al., 2013). Intrauterine transfusion of platelets is performed rarely, due to the high risk of fetal morbidity and mortality associated with an intrauterine transfusion of platelets, as well as the need to perform the procedure frequently due to the short life span of transfused platelets (Regan et al., 2019; Brojer et al., 2016; Espinoza et al., 2013).

Prevention of alloimmunization in pregnant women not already alloimmunized will prevent FNAIT and all its consequences for the fetus/neonate since anti-HPA-1a alloantibodies are the direct causative agent of FNAIT. Therefore, there is a need for a treatment that can prevent the maternal immune response that causes FNAIT.

### SEQUENCE LISTING

This application contains a Sequence Listing, which has been submitted electronically in ASCII format. The ASCII copy was created on Jun. 20, 2022, is named IPA_008_WO1_SL.txt, and is 9,816 bytes in size.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

Provided are methods and compositions for preventing maternal alloimmunization with HPA-1a and for preventing FNAIT caused by maternal alloimmunization with HPA-1a comprising a regimen for administration of an anti-HPA-1a antibody to a pregnant subject.

One embodiment is a method of preventing FNAIT caused by maternal alloimmunization with HPA-1a in a fetus of an HPA-1a-negative human subject, the method comprising parenterally administering to the subject multiple doses of a pharmaceutical composition comprising an effective amount of an anti-HPA-1a antibody, wherein the anti-HPA-1a antibody does not bind HPA-1b; wherein an initial dose of the pharmaceutical composition is administered between gestational weeks 10 and 16; wherein maintenance doses of the pharmaceutical composition are administered after the initial dose, at a regular dose interval throughout pregnancy; and wherein at least one dose is administered within 72 hours post-parturition. Also provided is a pharmaceutical composition comprising an effective amount of an anti-HPA-1a antibody for use in the method of preventing FNAIT caused by maternal alloimmunization with HPA-1a in a fetus of an HPA-1a-negative human subject.

Another embodiment is a method of preventing alloimmunization with HPA-1a in a subject, wherein the subject is an HPA-1a-negative pregnant woman, the method comprising parenterally administering to the subject multiple doses of a pharmaceutical composition comprising an anti-HPA-1a antibody, wherein the anti-HPA-1a antibody does not bind HPA-1b; wherein an initial dose of the pharmaceutical composition is administered between gestational weeks 10 and 16 of pregnancy; wherein maintenance doses of the pharmaceutical composition are administered after the initial dose, at regular dose intervals throughout pregnancy; and wherein at least one dose is administered within 72 hours post-parturition. Also provided is a pharmaceutical composition comprising an effective amount of an anti-HPA-1a antibody for use in the method of preventing alloimmunization with HPA-1a in a subject, wherein the subject is an HPA-1a-negative pregnant woman.

In certain embodiments, the subject is HLA-DRB3*01:01 positive.

In one embodiment, the anti-HPA-1a antibody is a polyclonal antibody. In a particular embodiment, the pharmaceutical composition is anti-HPA-1a gamma globulin. In one embodiment, the anti-HPA-1a antibody is a monoclonal antibody. In a particular embodiment, the monoclonal antibody is RLYB212.

In one embodiment, the pharmaceutical composition is administered via intravenous infusion. In a certain embodiment, the pharmaceutical composition is administered via subcutaneous injection. The pharmaceutical composition can be self-administered. In some embodiments, the pharmaceutical composition can be administered from a vial and syringe, a pre-filled syringe, a pen injector, or an autoinjector.

In some aspects of the invention, the regular dose interval is once weekly, twice weekly, or once every two weeks.

In one embodiment, the initial dose is the same as the maintenance dose. In another embodiment, the initial dose is higher than the maintenance dose.

In certain aspects, the Tₘₐₓ of the anti-HPA-1a antibody is immediately after administration of the initial dose. In other embodiments, the Tₘₐₓ of the initial dose of the anti-HPA-1a antibody is 5 to 15 days after administration. In certain aspects, a maintenance doses are administered weekly or biweekly after administration of the initial dose. In some embodiments, a peak concentration (Cₚ) of the anti-HPA-1a antibody is achieved 3-7 days after each maintenance dose. In a particular embodiment, the Tₘₐₓ of the anti-HPA-1a antibody is 3-7 days after the final maintenance dose, preferably 4-6 days or about 5 days after the final maintenance dose.

In some embodiments, the C_{threshold} of the anti-HPA-1a antibody is 0.3-0.7 IU/mL. In a particular embodiment, the C_{threshold} of the anti-HPA-1a antibody is 0.5 IU/mL or about 8.5 ng/mL.

In particular embodiments, the initial dose of the anti-HPA-1a antibody is 5-400 μg or 50-350 μg. In certain embodiments, the maintenance dose of the anti-HPA-1a antibody is 5-400 μg or 5-150 μg.

### BRIEF DESCRIPTION OF THE DRAWINGS

FIG. 1 shows the percentage of transfused platelets remaining in circulation of participants (n=8) administered anti-HPA-1a gamma globulin (study drug) or placebo, which occurred 60 minutes after transfusion with HPA-1a positive (and HLA-A2 positive) platelets. The data was normalized to peak (100%) for samples collected 15 minutes prior to administration of the study drug at 0 minutes. Each line represents a different subject. X-axis is not to scale.

FIG. 2A-2B show dose-dependent lysis of Chinese hamster ovary (CHO) cells (FIG. 2A) and HUVEC cells FIG. 2B) in the presence of monoclonal antibody 26.4 (mAb 26.4). Lysis was not observed in cells treated with effector-less IgG2/4 mAb, ZB002 or a non-binding IgG control, ZB007.

FIG. 3A-3D show binding isotherms of RLYB211 and RLYB212 to human and mouse platelets expressing HPA-1 alloantigens (FIG. 3A, 3B). Washed human platelets from HPA-1a/a and HPA-1b/b individuals were incubated with serially diluted RLYB211, RLYB212 or normal human IgG (NHIgG) and incubated for 1 hour at room temperature. Bound antibodies were detected using a 1/200 dilution of FITC-conjugated goat anti-human IgG. Note the specificity of RLYB211 and RLYB212 for the HPA-1a alloantigen. In FIG. 3C, platelets from the indicated species and having the indicated phenotypes were incubated with normal human IgG or RLYB211. In addition to the specificity of RLYB211 for HPA-1a expressed on GPIIIa on the surface of human platelets, RLYB211 is also specific for the APLDQ form of mouse GPIIIa on the surface of humanized, but not on wild-type, mouse platelets. In FIG. 3D, APLDQ platelets were incubated with increasing concentrations of polyclonal RLYB211 or the monospecific monoclonal antibody, RLYB212, and antibody binding quantified using flow cytometry to generate saturation-binding curves. Addition of ˜4 IU/mL of either antibody is well below saturation, and results in occupancy of <10% of the available GPIIb-IIIa receptors.

FIG. 4A-4B show that RLYB211 and RLYB212 induce clearance of circulating HPA-1a-positive murine platelets. Platelets isolated from APLDQ-positive C56BL/6 mice were labeled with the cell tracker dye CMFDA and were transfused (1×10⁸ CMFDA-labeled platelets/mouse) into wild-type Balb/c female mice. One hour after transfusion, ZB007 (1.34 mg), normal human IgG (2 mg), RLYB211, or RLYB212 at the indicated concentrations were introduced by tail vein injection. Blood samples were collected from the submandibular vein of the recipients following platelet transfusion but before antibody injection, and at 5 and 24 hours post antibody injection. The percentage of CMFDA-labeled platelets remaining in the circulation was determined by flow cytometry. The survival of transfused CMFDA-labeled APLDQ platelets in wild-type Balb/c female mice at 5 and 24 hours post antibody injection is shown. The survival of the transfused labeled platelets was calculated by the percentage of the remaining CMFDA⁺ platelets divided by the beginning percentage of CMFDA⁺ platelets. Data are the quantification of five separate experiments, shown as mean±SEM, N=5 per group. *P<0.05; **P<0.01, versus normal human IgG as analyzed by the Mann-Whitney test. ns, not significant.

FIG. 5A-5B show that administration of HPA-1a-specific antibodies prevents HPA-1a exposure-induced FNAIT. _Female wild-type BALB/c mice either received 2 mg of normal human IgG, 0.25, 1.0, 4 IU/ml of RLYB211 (FIG. 5A) or 1.34 mg ZB007 (normal human IgG), 1.2, 4.0, 12.0, 40.0 IU/ml of RLYB212 (FIG. 5B) one hour before transfusion of 1×10⁸ APLDQ murine platelets. Blood was collected at the indicated time points, and the antibodies present that were reactive against APLDQ platelets were measured by flow cytometry. Results are reported as median fluorescence intensity (MFI) (mean±SEM, n=8-10/group). Prevention of HPA-1a-induced alloimmunization is dose-dependent, with nearly complete protection beginning at 1 IU/ml of polyclonal RLYB211 and at 4 IU/ml of monoclonal RLYB212. *P<0.05; **P<0.01, ***P<0.001 versus normal human IgG as analyzed by the Mann-Whitney test. ns, not significant.

FIG. 6A-6D show that repeat administration of HPA-1a-specific antibodies provides sustained protection from HPA-1a exposure-induced FNAIT. In FIG. 6A, female wild-type BALB/c mice that had been challenged three weeks earlier with APLDQ platelets in the presence or absence of RLYB211 were re-challenged at Day 21 with 1×10⁸ APLDQ platelets with or without prior tail vein injection of additional RLYB211 or non-binding human IgG. Blood was collected at the indicated time points, and the maternal antibodies present that were reactive against APLDQ platelets were measured by flow cytometry. Results are reported as median fluorescence intensity (MFI) (mean±SEM, n=8-10/group). In FIG. 6B-6D, BALB/c female mice that had been challenged twice with APLDQ platelets in the presence or absence of RLYB211 were then bred at Day 35 with APLDQ males. About 21 days later, pups were born and their platelet counts were determined within 48 hours of birth (mean±SEM, n=44-69/group) (FIG. 6B). APLDQ-reactive maternal antibody levels present in the dam (mean SEM, n=6-9/group) (FIG. 6C) and in the pups (FIG. 6D) (mean±SEM, n=44-69/group) were then determined by flow cytometry. Platelet counts in pups born to female mice that had been treated with either no antibody or normal human IgG served as controls, and ranged from 3.5-5.5×10⁸/ml. Protection was dose-dependent, beginning at ˜1 IU/ml RYLB211. Significance was determined using the Mann-Whitney test. p<0.01 (**), p<0.001 (***), p<0.0001(****).

FIG. 7 shows one embodiment of the invention. Depicted is the predicted plasma concentration profile of a monoclonal anti-HPA-1a antibody over 91 days (13 weeks). Subcutaneous administration of RLYB212 at an initial loading dose of 0.29 mg, followed by biweekly (Q2W) repeat/maintenance doses of 0.1 mg (dashed line), is compared with a single administration of 0.21 mg RLYB212 (dotted line) or repeated administrations of the maintenance dose of 0.1 mg (dashed line). C_{threshold} for this regimen (25 ng/mL/˜1.48 IU/mL) is denoted by the solid horizontal line. Cₚ=peak concentration; LD=loading dose; MD=maintenance dose.

### DETAILED DESCRIPTION OF THE INVENTION

The practice of the present invention can employ, unless otherwise indicated, conventional techniques of pharmaceutics, formulation science, protein chemistry, cell biology, cell culture, molecular biology, microbiology, recombinant DNA, immunology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### I. Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes ±10% of the recited value. For example, a concentration of about 1 mg/mL includes 0.9 mg/mL to 1.1 mg/mL. Likewise, a concentration range of about 1% to 10% (w/v) includes 0.9% (w/v) to 11% (w/v).

The terms “polypeptide,” “peptide,” and “protein” are used interchangeably to refer to polymers of amino acids of any length, and their salts. The polymer can be linear or branched, can comprise modified amino acids, and can be interrupted by non-amino acids. Except where indicated otherwise, e.g., for the abbreviations for the uncommon or unnatural amino acids set forth herein, the three-letter and one-letter abbreviations, as used in the art, are used herein to represent amino acid residues. Groups or strings of amino acid abbreviations are used to represent peptides. Except where specifically indicated, peptides are indicated with the N-terminus of the left and the sequence is written from the N-terminus to the C-terminus.

A “polynucleotide,” as used herein can include one or more “nucleic acids,” “nucleic acid molecules,” or “nucleic acid sequences,” and refers to a polymer of nucleotides of any length, and includes DNA and RNA. The polynucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

An “isolated” molecule is one that is in a form not found in nature, including those which have been purified.

A “label” is a detectable compound that can be conjugated directly or indirectly to a molecule, so as to generate a “labeled” molecule. The label can be detectable on its own (e.g., radioisotope labels or fluorescent labels), or can be indirectly detected, for example, by catalyzing chemical alteration of a substrate compound or composition that is detectable (e.g., an enzymatic label) or by other means of indirect detection (e.g., biotinylation).

The human amino acid sequence of GPIIIa (integrin β3) is set forth in GenBank accession no. AAA52589.1, which includes a 26-amino acid signal peptide. HPA-1 is a polymorphism at position 33 of the mature integrin β3 chain. Individuals who have a Leu at position 33 in one or more copies of ITGB3 (i.e., the gene that encodes integrin β3) or in any of their integrin β3 are “HPA-1a positive,” “positive for HPA-1a,” “HPA-1a,” or “HPA-1ab,” while individuals who do not have a Leu at position 33 (e.g., have a Pro at position 33) in all copies of ITGB3 or in all of their integrin β3 are “HPA-1a negative” or “negative for HPA-1a.”

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin” are used interchangeably herein.

A typical antibody is composed of two identical pairs of polypeptide chains, each pair having one “heavy” chain and one “light” chain. Each chain is comprised of a variable region, which forms the antibody binding site, and a constant region, which can mediate the binding of the antibody to host tissues or factors. Immunoglobulin molecules can be divided into classes depending on the constant region of the heavy chain. The classes are immunoglobulin gamma (IgG), immunoglobulin mu (IgM), immunoglobulin delta (IgD), immunoglobulin epsilon (IgE), and immunoglobulin alpha (IgA). The heavy chain constant regions differ structurally and antigenically among the subclasses. IgG is the main type of antibody found in blood and extracellular fluid, and it plays a central role in the humoral immune response.

A “monoclonal antibody” (mAb) refers to a homogeneous antibody population that is involved in the highly specific recognition and binding of a single antigenic determinant (epitope). “Polyclonal antibodies” are a mixture of monoclonal antibodies directed against different epitopes of the same antigen. The term “monoclonal” can apply to full-length monoclonal antibodies, as well as to antigen-binding fragments, fusion proteins comprising an antigen-binding region, and any other modified immunoglobulin molecule comprising an antigen recognition site.

The term “antigen-binding fragment” refers to a portion of an intact antibody comprising the complementarity determining regions of the antibody. Examples of antigen-binding fragments include Fab, Fab′, F(ab′)2, and Fv fragments, linear antibodies, single chain antibodies (e.g., ScFvs), and multi-specific antibodies formed from antibody fragments.

“Binding affinity” generally refers to the strength of the sum total of non-covalent interactions between a single binding site of a molecule and its binding partner (e.g., a receptor and its ligand, an antibody and its antigen, two monomers that form a dimer, etc.). Unless indicated otherwise, as used herein, “binding affinity” refers to intrinsic binding affinity which reflects a 1:1 interaction between members of a binding pair. The affinity of a molecule X for its partner Y can generally be represented by the dissociation constant (K_{D}). Affinity can be measured by common methods known in the art, including those described herein. Low-affinity binding partners generally bind slowly and tend to dissociate readily, whereas high-affinity binding partners generally bind faster and tend to remain bound longer.

The affinity or avidity of a molecule for its binding partner can be determined experimentally using any suitable method known in the art, e.g., flow cytometry, enzyme-linked immunosorbent assay (ELISA), or radioimmunoassay (RIA), or kinetics (e.g., KINEXA® or BIACORE™ or OCTET® analysis). Direct binding assays as well as competitive binding assay formats can be readily employed. (See, e.g., Berzofsky et al., “Antibody-Antigen Interactions,” in Fundamental Immunology, Paul, W. E., ed., Raven Press: New York, N.Y. (1984); Kuby, Immunology, W. H. Freeman and Company: New York, N.Y. (1992)). The measured affinity of a particular binding pair interaction can vary if measured under different conditions (e.g., salt concentration, pH, temperature). Thus, measurements of affinity and other binding parameters (e.g., K_{D} or Kd, Kₒₙ, K_{off}) are made with standardized solutions of binding partners and a standardized buffer, as known in the art.

An “active agent” is an ingredient that is intended to furnish biological activity. The active agent can be in association with one or more other ingredients. An active agent that is a peptide can also be referred to as an “active peptide.”

The term “international unit” or “IU” is a unit of measurement of the amount of a substance as determined by its activity. The mass or volume that constitutes one international unit of a substance will vary based on the substance that is being measured. For anti-HPA-1a antibodies, the amount of anti-HPA1a antibodies in one IU is set to an international standard (Allen et al. 2005) adopted by the World Health Organization (see WHO International Standard: Anti-HPA-1a Standard (100 IU)).

As used herein, “WHO Standard” refers to the WHO International Standard of anti-HPA-1a antibodies, prepared by pooling human plasma collected from six donors immunized against HPA-1a (see WHO International Standard: Anti-HPA-1a Standard (100 IU)).

An “effective amount” of an active agent is an amount sufficient to carry out a specifically stated purpose.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g. acetate, phosphate, or citrate buffer), a surfactant (e.g. polysorbate), a stabilizing agent (e.g. polyol or amino acid), a preservative (e.g. sodium benzoate), and/or other conventional solubilizing or dispersing agents.

A “subject” or “individual” or “animal” or “patient” or “mammal,” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

The terms “alloimmune response” or “alloimmunization” is an immune response to non-self antigens that are from the same species. As a result, the body produces antibodies against the non-self antigens.

With respect to the presence of a cell type, such as platelets, the terms “clear,” “clearance,” “eliminate,” and “elimination” are used interchangeably, and refer to achieving an undetectable level of the cell type. Detection of the cell type can be carried out by known methods, including, for example, immunohistochemistry or flow cytometry, such as fluorescence-activated cell sorting (FACS).

The terms “inhibit,” “block,” and “suppress” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

“Prevent” or “prevention” refers to prophylactic or preventative measures that prevent and/or slow the development of a targeted pathologic condition or disorder. Thus, those in need of prevention include those at risk of or susceptible to developing the disorder.

“Pharmacokinetics” or “PK” refers to the study of how an administered agent is processed by the body of a subject. PK determinations include how the agent enters the blood circulation (absorption), is dispersed or disseminated throughout the fluids and tissues of the body (distribution), is recognized and transformed by the body (metabolism), and is removed from the body (excretion). The agent can be an active agent, e.g., a therapeutic antibody. Pharmacokinetics can be evaluated using various metrics, many of which are calculated based on the quantity of the agent in the body (e.g., in the plasma) at various time points following the administration of the agent.

“Cₘₐₓ” or “Cₚ” is the peak plasma concentration of an agent after administration. “Cₚₛₛ” is the trough plasma concentration of an agent at steady-state. “C_{threshold}” is the target plasma concentration (exposure threshold) of an agent.

“Steady state” is achieved when the plasma concentration of an agent is maintained at a therapeutically effective level by administration of regular doses of the agent to balance the amount of drug being cleared. Once steady state is reached, the plasma concentration of the agent ranges from a peak (Cₘₐₓ) to a trough (Cₘᵢₙ) concentration.

Time after administration is measured from T₀, which is the time that administration of a single dose of the agent is administered. “Tₘₐₓ” refers to the time of after administration of the agent (T₀) to reach maximum plasma concentration (Cₘₐₓ or Cₚ) of the agent. “T_{1/2}” refers to the half-life of the agent, i.e., the time required for the concentration of the agent to reach half of its original value.

### II. Administration and Methods of Prevention

Mismatch between fetal and maternal IPA-1 alloantigens, where an HPA-1a-negative woman carries a fetus having a paternally-inherited HPA-1a allotype, can lead to maternal production of anti-HPA-1a antibodies. These antibodies can traverse the placenta, bind fetal platelets, and accelerate platelet destruction, causing fetal neonatal alloimmune thrombocytopenia (FNAIT). There is currently no prophylactic treatment for FNAIT.

A subject “at risk” of having an FNAIT pregnancy is an HPA-1a negative woman who becomes pregnant with an HPA-1a positive fetus. Women at “higher FNAIT risk” are HPA-1a negative, indicating FNAIT risk, and are HLA-DRB3*01:01 positive; indicating ˜25-fold higher alloimmunization risk compared to those without this human leukocyte antigen (HLA) allele.

Administration of polyclonal or monoclonal anti-HPA-1a antibodies to an HPA-1a negative mother has been contemplated as a potential prophylaxis for FNAIT (Kjeldsen-Kragh et al., 2012; Tiller et al., 2012; Eksteen et al., 2015). The prior art identified the 72 hours before and after birth as the critical timeframe for administration of anti-HPA-1a antibodies, because maternal alloimmunization was believed to be largely the result of fetal platelets entering the mother's circulation in association with delivery (Kjeldsen-Kragh et al., 2012; Tiller et al., 2012; Eksteen et al., 2015). However, in addition to being expressed on the membrane of platelets, integrin β3 is also expressed on the membrane of other cell types, creating additional sources of potential antigenic stimulation for the maternal immune system (Zhou et al., 1997).

Contrary to the theory that a single administration of anti-platelet antibodies after antigenic challenge would prevent alloimmunization, the present invention provides a multi-dose administration regimen in which a composition comprising an antibody specific for HPA-1a is administered to a woman at risk for FNAIT early in pregnancy, before exposure to the HPA-1a antigen, and is continued throughout the course of the pregnancy. The anti-HPA-1a antibody is administered at doses well below the threshold known to cause adverse clinical sequelae in the fetus or neonate, can safely and effectively prevent maternal alloimmunization.

One embodiment is a method of preventing FNAIT caused by maternal alloimmunization with HPA-1a in a fetus of an HPA-1a-negative subject, the method comprising parenterally administering to the subject multiple doses of a pharmaceutical composition comprising an effective amount of an anti-HPA-1a antibody, wherein the anti-HPA-1a antibody does not bind HPA-1b; wherein an initial dose of the pharmaceutical composition is administered between gestational weeks 10 and 16; wherein maintenance doses of the pharmaceutical composition are administered after the initial dose, at a regular dose interval until delivery; and wherein at least one dose is administered within 72 hours post-parturition. Also included is a pharmaceutical composition comprising an effective amount of an antibody specific for HPA-1a for use in the method.

Another embodiment is a method of preventing alloimmunization with HPA-1a in a subject, wherein the subject is an HPA-1a-negative pregnant woman, the method comprising parenterally administering to the subject multiple doses of a pharmaceutical composition comprising an anti-HPA-1a antibody, wherein the anti-HPA-1a antibody does not bind HPA-1b; wherein an initial dose of the pharmaceutical composition is administered between gestational weeks 10 and 16; wherein maintenance doses of the pharmaceutical composition are administered after the initial dose, at a regular dose interval until delivery; and wherein at least one dose is administered within 72 hours post-parturition. Also included is a pharmaceutical composition comprising an effective amount of an antibody specific for anti-HPA-1a for use in the method.

Gestational age is determined by known methods, including menstrual history, clinical examination, and/or ultrasonography. “Delivery” and “parturition” are used interchangeably in reference to childbirth.

“Initial dose,” “loading dose,” and “induction dose” are used interchangeably and refer to the first dose of the pharmaceutical composition comprising an anti-HPA-1a antibody administered to the subject. “Maintenance dose” and “repeat dose” are used interchangeably and refer to the doses of the pharmaceutical composition administered to the subject subsequent to the initial dose.

The pharmaceutical composition comprising an anti-HPA-1a antibody is administered parenterally. Parenteral routes of administration include intravenous, intramuscular, intraperitoneal, intrathecal, and subcutaneous. In a preferred embodiment, the pharmaceutical composition is administered subcutaneously. The pharmaceutical composition can be administered, for example, via a vial and syringe, a pre-filled syringe, a pen injector, or an autoinjector. The pharmaceutical composition can be self-administered. “Self-administration” means that the pharmaceutical composition is administered by the subject, and can also include administration by someone else, such as a family member or friend.

Maintenance doses of the pharmaceutical composition are administered at a regular dose interval, meaning that the time between doses is fixed. In one embodiment, the pharmaceutical composition is administered about every 48 hours or about every 72 hours or about every 96 hours or about every 5 days or about every 6 days. In another embodiment, the pharmaceutical composition is administered once weekly (QW), or twice weekly (BIW), or once every two weeks (Q2W). At least one dose is administered within about 72 hours of delivery. Subsequent post-parturition doses can be administered about 4, 5, or 6 days after delivery, or about 1, 2, 3, 4, 5, 6, 7, or 8 weeks after delivery.

In some embodiments, the initial dose of the anti-HPA-1a antibody is the same as the maintenance dose, while in other embodiments, the initial dose of the anti-HPA-1a antibody is higher than the maintenance dose. For example, the initial dose can be about 2, 3, 4, 5, 6, 7, 8, 9, or 10 times the maintenance dose. In preferred embodiments, the initial dose is 5 or 6 times a QW maintenance dose or 3 or 4 times a Q2W maintenance dose. In one embodiment, the initial dose of the anti-HPA-1a antibody is about 5-400 μg or about 30-350 μg or about 60-270 μg or about 90-180 μg. In one embodiment, the maintenance dose of the anti-HPA-1a antibody is about 5-400 μg or about 10-200 μg or about 15-120 μg or about 20-90 μg or about or about 25, 30, 35, 40, 45, 50, or 55 μg. In one embodiment in which the anti-HPA-1a antibody is RLYB212, the initial dose is about 120-270 μg and the maintenance dose is about 15-60 μg. In a particular embodiment in which the anti-HPA-1a antibody is RLYB212, the initial dose is about 180 μg and the maintenance dose is about 30 μg QW or about 60 μg Q2W. In another embodiment in which the anti-HPA-1a antibody is RLYB212, the initial dose is about 270 μg and the maintenance dose is about 45 μg QW or about 90 μg Q2W.

The methods of the invention involve administration of an amount of antibody specific for HPA-1a that is effective to prevent maternal alloimmunization or FNAIT caused by maternal alloimmunization. An effective amount of a given anti-HPA-1a antibody can be determined, for example, by the ability of the antibody to clear HPA-1a positive platelets in the circulation of an HPA-1a-negative subject. Preferably, HPA-1a positive platelets are cleared in a subject within about 6-24 hours of administering the anti-HPA-1a antibody, more preferably within about 2 or 3 hours of administering the anti-HPA-1a antibody. In certain embodiments, HPA-1a positive platelets are cleared in a population of subjects within a mean of about 6-24 hours of administering the anti-HPA-1a antibody to the subjects, more preferably within about 2 or 3 hours of administering the anti-HPA-1a antibody.

In certain embodiments, the effective amount is determined by the dose required to achieve a particular target plasma concentration (C_{threshold}) of the anti-HPA-1a antibody. Preferably, the C_{threshold} is about 0.3-3.0 IU/mL, or about 0.3-0.7 IU/mL, or about 6-10 ng/mL. In one embodiment, the C_{threshold} is 0.5 IU/mL. In one embodiment, the C_{threshold} is 51 ng/mL. In an embodiment in which the anti-HPA-1a antibody is RLYB212, the C_{threshold} is 8.0-9.0 ng/mL. In a particular embodiment, the C_{threshold} is 8.5 ng/mL. Concentration of an anti-HPA-1a antibody can be determined, for example, using a monoclonal antibody-specific immobilization of platelet antigens (MAIPA) assay (Kiefel et al., 1987; Campbell et al., 2007) or other ELISA-based method.

In certain embodiments, the anti-HPA-1a antibody prevents FNAIT caused by maternal alloimmunization by outcompeting alloantibody binding to fetal platelets. For example, the anti-HPA-1a antibody can be an effectorless antibody. The C_{threshold} in such embodiments can be 10,000, 20,000, 30,000, 40,000, or 50,000 IU/mL.

In some embodiments, the antibody drives clearance of fetal-derived antigen but does not bind neonatal Fc receptor (FcRn), and therefore, does not cross the placenta. In such embodiments, there is no risk to the fetus of pathological effects from the treatment, because there is no fetal exposure to the anti-HPA-1a antibody. The C_{threshold} in such embodiments can be 10,000, 20,000, 30,000, 40,000, or 50,000 IU/mL.

In embodiments in which the anti-HPA-1a antibody is administered intravenously, the Tₘₐₓ is at the end of infusion. In some embodiments in which the anti-HPA-1a antibody is administered subcutaneously, the Tₘₐₓ of the anti-HPA-1a antibody is about 5-15 days, or about 8-12 days or about 9-11 days after administration of the initial dose. In embodiments wherein the initial dose is higher than the maintenance dose, the Tₘₐₓ can be achieved, for example, after the first maintenance dose, or after a subsequent maintenance dose. In one embodiment, the Tₘₐₓ of the anti-HPA-1a antibody is about 3-7 days after administration of the final maintenance dose, for example, about 5 days after the final maintenance dose. Each maintenance dose will achieve a Cₘₐₓ before falling to a trough concentration (Cₘᵢₙ). In some embodiments, Cₘₐₓ of the anti-HPA-1a antibody is reached 3-7 days after each maintenance dose. In one embodiment, Tₘₐₓ of the anti-HPA-1a antibody for an administration regimen of the invention is 3-7 days, preferably about 5 days, after administration of the final maintenance dose. In certain embodiments, at steady state, the plasma concentration of anti-HPA-1a antibody is between about 6.5 ng/mL and 8.5 ng/mL.

### Antibodies and Compositions

The administration regimens provided herein can employ any anti-HPA-1a antibody that binds specifically to HPA-1a, such as a monoclonal antibody, a polyclonal antibody, or an antigen-binding fragment thereof. Anti-HPA-1a antibodies administered in a regimen of the invention are “specific for HPA-1a,” which means that they do not display detectable binding to HPA-1b.

The polyclonal antibody can be “anti-HPA-1a gamma globulin,” which refers to a preparation produced from pooled plasma of donors with anti-HPA-1a antibodies. A polyclonal antibody preparation can also be produced from the plasma of a single donor with anti-HPA-1a antibodies. In one embodiment, the donor is an HPA-1a negative subject who has been alloimmunized with HPA-1a, for example, as a result of a previous pregnancy with an HPA-1a-positive fetus. In another embodiment, the donor is an HPA-1a negative subject who has been deliberately immunized with HPA-1a positive platelets or with a purified or recombinant preparation HPA-1a antigen. The preparation contains the total IgG from the pooled source plasma. See, e.g., U.S. Pat. No. 9,834,613.

Examples of monoclonal anti-HPA-1a antibodies include, for instance, mAb 26.4 (Eksteen et al., 2015) and RLYB212, both of which are human monoclonal antibodies that bind specifically to the HPA-1a isoform of integrin β3 and do not display detectable binding to the HPA-1b isoform of either recombinant or native integrin β3. RLYB212 differs from mAb 26.4 by a single amino acid substitution in the heavy chain, with the replacement of methionine at position 96 with valine to eliminate a potential site of oxidation. The CDRs of mAb 26.4 and RLYB212, as designated by the International ImMunoGeneTics (IMGT) method (Lefranc et al., 2003), are set forth in SEQ ID NO: 3-8.

The anti-HPA-1a antibody is formulated in a pharmaceutical composition. The pH of the composition can be between about 3.0 and 8.0. In certain embodiments, the pH is between about 4.0 and 7.0, or between about 5.0 and 6.5. In one embodiment, the pH is about 6.3.

The pharmaceutical composition can comprise one or more carriers, diluents, excipients, or other additives. For example, the composition can comprise one or more stabilizing agents (e.g., dextran 40, glycine, lactose, mannitol, trehalose, maltose), one or more buffers (e.g., acetate, citrate, histidine, lactate, phosphate, Tris), one or more pH adjusting agents (e.g., hydrochloric acid, nitric acid, potassium hydroxide, sodium hydroxide), one or more surfactants (polysorbate, sodium lauryl sulfate, polyethylene glycol-fatty acid esters, lecithins), and/or one or more diluents (e.g., water, physiological saline). In certain embodiments, the composition does not comprise mercury. In certain embodiments, the composition does not comprise a preservative.

In one embodiment, the pharmaceutical composition comprises anti-HPA-1a gamma globulin, maltose, and polysorbate 80. In one embodiment, the pharmaceutical composition comprises RLYB212 succinate, arginine, polysorbate 80, and water for injection.

### IV. Methods of Preparation

Monoclonal antibodies can be prepared by methods known in the art. For example, an anti-HPA-1a antibody can be prepared from memory B cells isolated from an HPA-1a alloimmunized subject according to the method described by Eksteen et al. (2015). Alternatively, a recombinant anti-HPA-1a antibody, for example, mAb 26.4 or RLYB212, can be expressed in host cells. In another embodiment, anti-HPA-1a antibodies can be raised in mice or other mammals immunized with human HPA-1a, produced using hybridoma technology (Kohler et al., 1975), and preferably humanized.

Polyclonal antibodies can be prepared by producing a mixture of two or more monoclonal antibodies. Alternatively, polyclonal antibodies can be prepared from plasma of one or more donors with anti-HPA-1a antibodies. The manufacturing process can comprise purification of IgG from source plasma containing antibodies to HPA-1a, clearance of viruses from the purified IgG, and concentration of the purified IgG. Purification of the IgG can be performed using anion-exchange chromatography, although other suitable techniques can be used, such as alcohol fractionation and polyethylene glycol (PEG) precipitation.

Viral clearance in purified IgG can be performed by virus removal, for example, by phase partitioning or PEG precipitation, affinity chromatography, ion exchange or gel exclusion chromatography, filtration, etc.; by virus inactivation, for example, by cold ethanol fractionation, heating, solvent/detergent, exposure to an acidic environment, etc.; or by a combination thereof. In some embodiments, viral clearance is performed by nanofiltration and exposure of the purified IgG to a solvent detergent, such as tri-n-butyl phosphate. Use of a solvent detergent to clear viruses from purified IgG can be followed by removal of the solvent detergent, for example, by reverse-phase chromatography.

Following viral clearance, the purified IgG can be concentrated using, for example, ultrafiltration. In addition, diafiltration can be used to remove microsolutes such as salts from the preparation.

Additional steps can be included. For instance, prior to purification, plasma can be diluted and dextran sulphate can be added to plasma to remove lipids. Plasma from different sources (e.g., from different persons) can be pooled together. After viral clearance, a step can be performed to reduce procoagulant factors, such as Factor XI and activated Factor XI, for example, by affinity chromatography.

### EXAMPLES

Embodiments of the present disclosure can be further defined by reference to the following non-limiting examples. It will be apparent to those skilled in the art that many modifications, both to materials and methods, can be practiced without departing from the scope of the present disclosure.

### Example 1. Clearance of HPA-1a-Positive Platelets by Polyclonal Anti-HPA-1a Antibodies

Anti-HPA-1a gamma globulin is a preparation of polyclonal anti-HPA-1a antibodies, produced from pooled plasma of donors who have been alloimmunized with HPA-1a as a result of a previous pregnancy with an HPA-1a-positive fetus. The preparation contains the total IgG from the pooled source plasma. The ability of anti-HPA-1a gamma globulin to target and eliminate HPA-1a-positive platelets from the bloodstream of HPA-1a-negative subjects was tested.

To ensure HLA discrepancy between platelet donor and recipient, which is necessary for detecting the transfused platelets by flow cytometry, donor platelets were positive for HLA-A2, which is not expressed on recipient platelets. On Day 1, the participants received a transfusion of HPA-1a positive (and HLA-A2 positive) platelets at a dose of 10×10⁹, which corresponds to the approximate number of platelets in 30 mL of fetal blood. Sixty minutes after completion of the transfusion, HPA-1a- and HLA-A2-negative subjects were administered 1,000 IU anti-HPA-1a gamma globulin or placebo (0.9% saline) through a peripheral venous catheter at approximately 10 mL/60 seconds.

Flow cytometry was used to directly assesses the survival of transfused platelets (Vetlesen et al, 2012). This method takes advantage of the discrepancy between donor and recipient HLA class I molecules that are expressed on the surface of platelets. By using fluorochrome-conjugated anti-HLA antibodies, it is possible to distinguish between populations of platelets with different HLA types.

Platelets were collected on Day 1 at the following timepoints:
15 min (+5 min) prior to transfusion;
15 min (+5 min) prior to study drug administration; and
10 min (+5 min), 20 min (+5 min), 30 min (+5 min), 40 min (+5 min), 50 min (+5 min), 1 hr (+5 min), 2 hr (10 min), 3 hr (15 min), 4 hr (15 min), and 24 hr (+1 hr) after study drug administration.
   In addition, a single draw of platelets occurred on Day 3 and, if transfused platelets were detected, on Day 7.

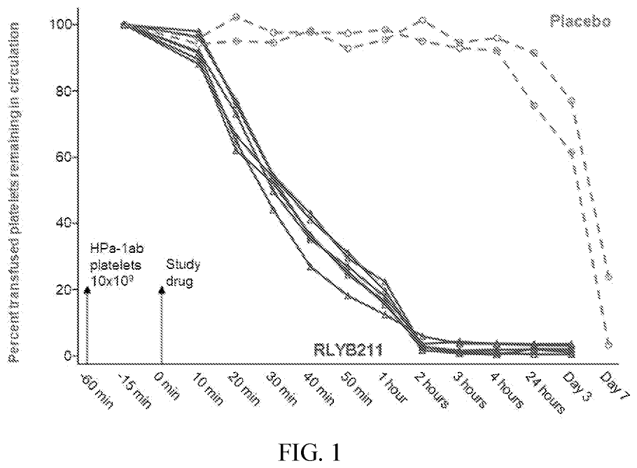

Administration of anti-HPA-1a gamma globulin markedly accelerated the clearance of the transfused platelets compared with placebo. In general, following administration of anti-HPA-1a gamma globulin, the percentage of transfused platelets remaining in circulation decreased to less than about 40% within one hour, and to less than about 10% within two hours (FIG. 1). In contrast, the percentage of transfused platelets remaining in circulation in participants administered placebo remained above about 90% through at least four hours. In subjects transfused with placebo, more than 50% of transfused platelets remained in circulation after three days, while transfused platelets could not be detected after three days in subjects transfused with anti-HPA-1a gamma globulin.

Overall, the half-life of mismatched platelets in subjects infused with anti-HPA-1a gamma globulin and subjects infused with placebo was 0.32 hours and 65.29 hours, respectively (p<0.001). Further, administration of anti-HPA-1a gamma globulin showed acceptable safety and tolerability with no serious adverse events and minimal adverse events observed.

These results demonstrate that administration of anti-HPA-1a gamma globulin at a dose of 1,000 IU effectively cleared transfused HPA-1a positive platelets from the circulation of the treated subjects.

### Example 2. Cellular Toxicity of Monoclonal Anti-HPA-1a Antibody in Cells Expressing Integrin αIIβ and αVβ3

Monoclonal antibody (mAb) 26.4 is a recombinant human immunoglobulin G1 monoclonal antibody that specifically binds to anti-HPA-1a. See, e.g., WO 2015/150417. mAb 26.4 binds integrin β3 in both integrin αIIbβ3 complexes expressed primarily on platelets (thrombin receptor), and integrin αvβ complexes expressed primarily on endothelial and trophoblastic cells (vitronectin receptor) (Eksteen et al., 2015).

A Chinese Hamster Ovary (CHO) cell line (αIIbβ3-CHO), engineered to stably express human integrin αIIβ3 (Baker et al., 1997), and human umbilical vascular endothelial cells (HUVEC), known to express integrin αVβ3 (Defilippi et al., 1991) were selected as the target cell for antibody-dependent cellular cytotoxicity (ADCC) after opsonization with mAb26.4. Primary human natural killer (NK) cells from two unrelated donors were selected as effector cells to mediate ADCC. Assay methods were as follows:
1. αIIbβ3-CHO, and HUVEC cells were washed in RPMI-1640 (without phenol red)+2% HiFBS and resuspended in RPMI-1640+10% HiFBS to 1×10⁶ cells/mL.
2. Cells were then stained with 5 μM calcein AM for 30 minutes at 37° C.
3. Excess calcein was removed by washing.
4. Cells were resuspended to 6.25×10⁴ cells/mL in RPMI-1640+10% HiFBS+probenecid (final assay concentration: 2.5 mM) and plated at 5000 cells/well in a 96-well V-bottom plate in triplicates for each condition.
5. Five-fold serially diluted mAb 26.4, Effector-less IgG2/4 mAb (ZB002), and a non-binding IgG control (ZB007) were added to the HUVEC or CHO cells (final starting concentration in the assay: 1 μg/mL).
6. After incubation with respective antibodies, NK cells (effector cells) were added to the target cells at a 5:1 effector to target (E:T) ratio. IL2 was added to the NK cells at 10 ng/mL, for the duration of the assay.
7. Cells were incubated for 4 hours at 37° C.
8. Media was removed from the plate and transferred to a 96-well clear bottom black plate.
9. Fluorescence intensity was measured at 485/530 nm.
10. Control target cells were also plated and treated with 2% Triton X-100 to determine the maximal fluorescence signal.

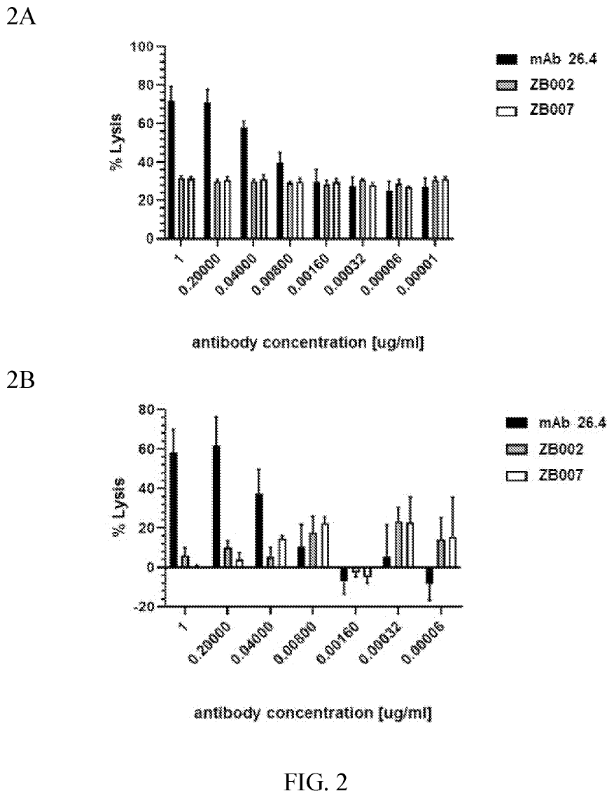

Dose-dependent cell lysis was observed for both αIIbβ3-CHO and HUVEC cells in the presence of mAb 26.4, while the IgG2/4 variant (ZB002) and non-binding IgG1 isotype control (ZB007) did not confer lytic activity (FIG. 2A-2B).

### Example 3. Relative Abilities of Polyclonal and Monoclonal Anti-HPA-1a Antibodies to Saturate HPA-1a Epitopes on Human and APLDQ Transgenic Mouse Platelets

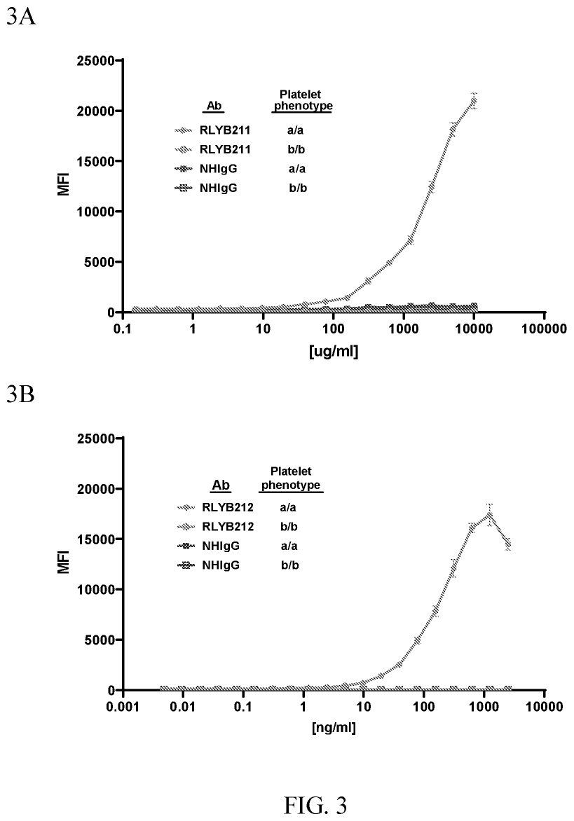

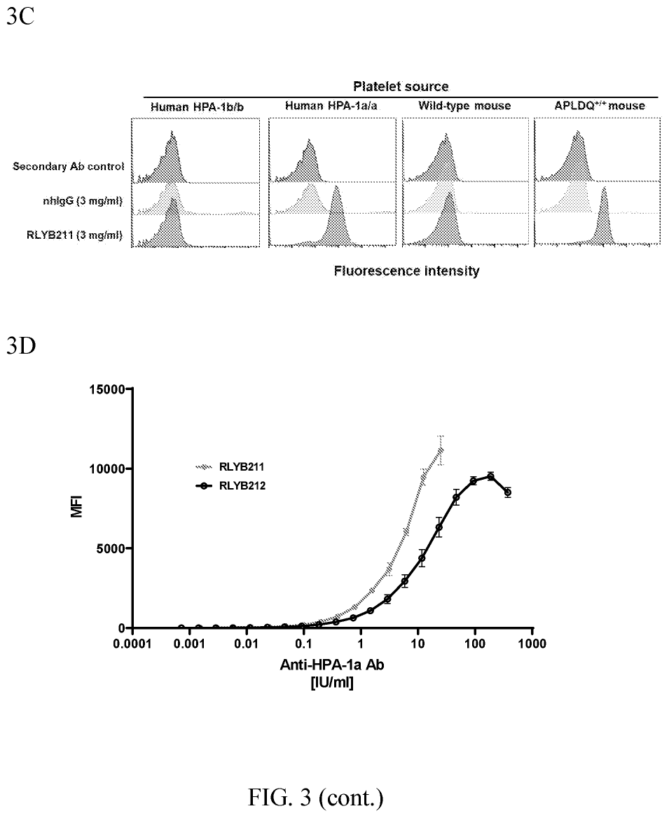

In a bi-allelic murine model of FNAIT (Zhi et al., 2019), the gene encoding murine integrin β3 was “humanized” through CRISPR-Cas9 gene editing to introduce the human HPA-1a epitope by replacing 5 murine amino acids with their human counterparts (Alanine-30, Proline-32, Leucine-33, Aspartic Acid-39, and Glutamine-470). Transgenic mice were humanized to express the HPA-1a epitope on a murine GPIIIa backbone (hereafter termed APLDQ GPIIIa), and used to demonstrate that the human anti-HPA-1a-specific mAb, 26.4, binds to APLDQ, but not wild-type, murine platelets (Zhi et al., 2018). RLYB212 was developed from mAb 26.4 and retains the parental binding characteristics. RLYB211 is a hyperimmune anti-HPA-1a polyclonal IgG preparation; however its ability to bind the APLDQ epitope has not been previously characterized. As shown in FIG. 3A, RLYB211 binds specifically to HPA-1^{a/a}, but not HPA-1^{b/b}, human platelets, and also selectively binds to APLDQ platelets (HPA-1a positive), but not to wild-type, murine platelets (FIG. 3B).

To assess the efficacy of RLYB211 and RLYB212, the potency of each antibody preparation was determined in a qualified ELISA assay for binding to HPA-1a. Binding potency was normalized against the WHO anti-HPA-1a standard and is represented as international units (IU). Concentration-binding isotherms were assessed for RLYB211 and RLYB212 (in IU/mL) against APLDQ homozygous platelets. The dynamic range of binding-response curve was comparable (within 2-fold) for each of these two antibodies (FIG. 3C). RLYB212 was able to fully saturate GPIIb-IIIa receptors at concentrations >˜100 IU/ml (FIG. 3D); however, receptor saturation with RLYB211 could not be established due to the low percentage of anti-HPA-1a specific antibodies in the polyclonal antibody (relative to total IgG).

### Example 4. RLYB211 and RLYB212 Eliminate Circulating HPA-1a-Positive Murine Platelets, Prevent Alloimmunization, and Rescue Neonatal Platelet Counts

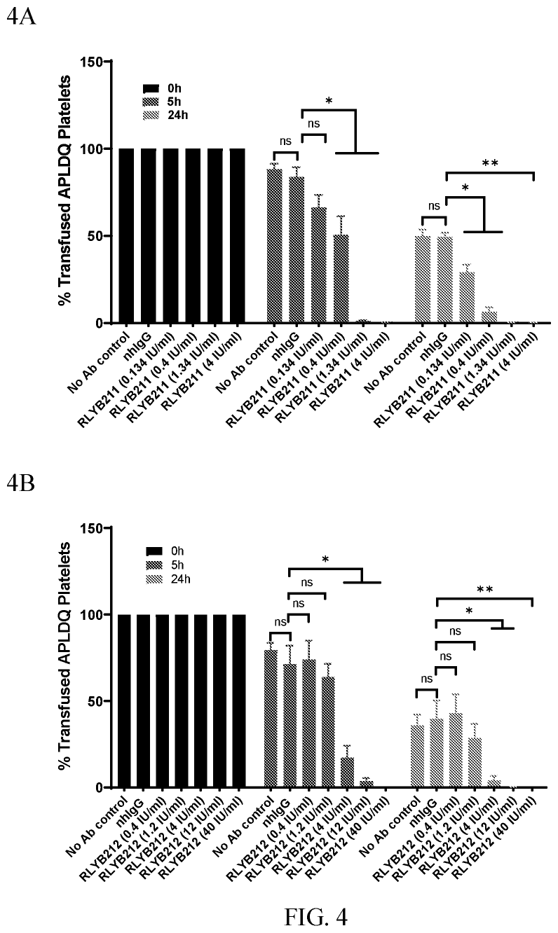

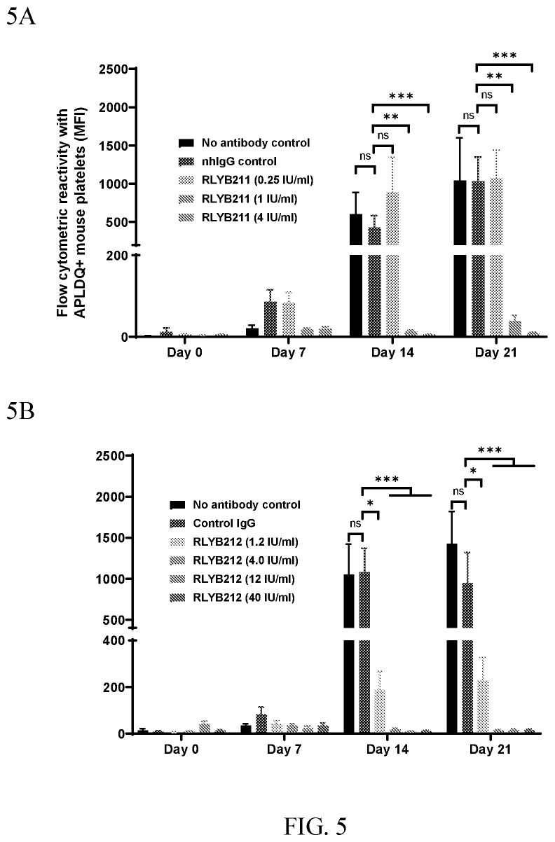

To evaluate the relative ability of either polyclonal or monoclonal HPA-1a-specific antibodies to remove foreign HPA-1a antigen from circulation, each antibody was administered by intravenous (IV) injection into wild-type BALB/c mice 1 hour after IV transfusion of 1×10⁸ CMFDA-labeled APLDQ-homozygous platelets. In three repeated experiments, doses estimated to yield exposures of approximately 1.34 to 4 IU/ml of either monoclonal RLYB212 or polyclonal RLYB211 were each effective at removing nearly all APLDQ-positive platelets from circulation within five hours (FIG. 4A, 4B). Similar doses of these reagents were also effective at preventing alloimmunization, as determined by measuring antibody titers two- and three-weeks following exposure to murine HPA-1a-positive platelets (FIG. 5A, 5B).

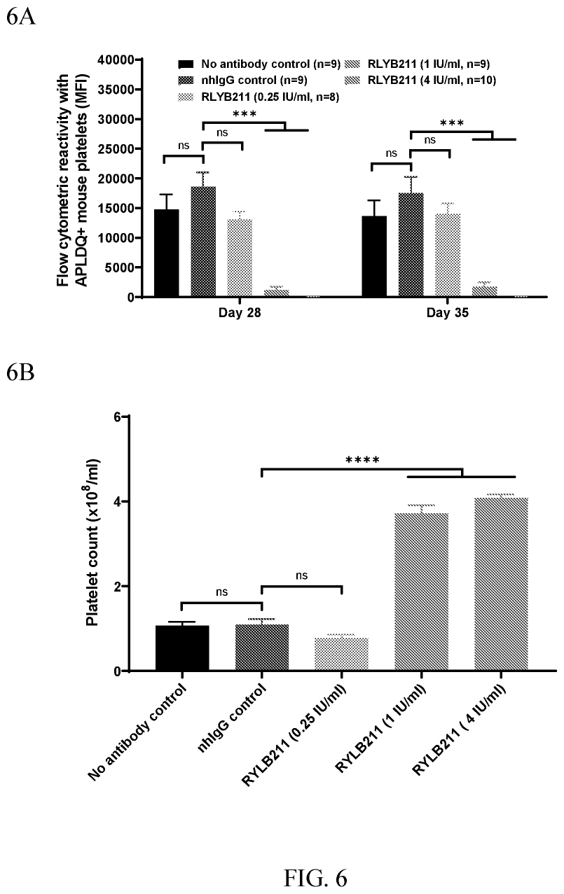

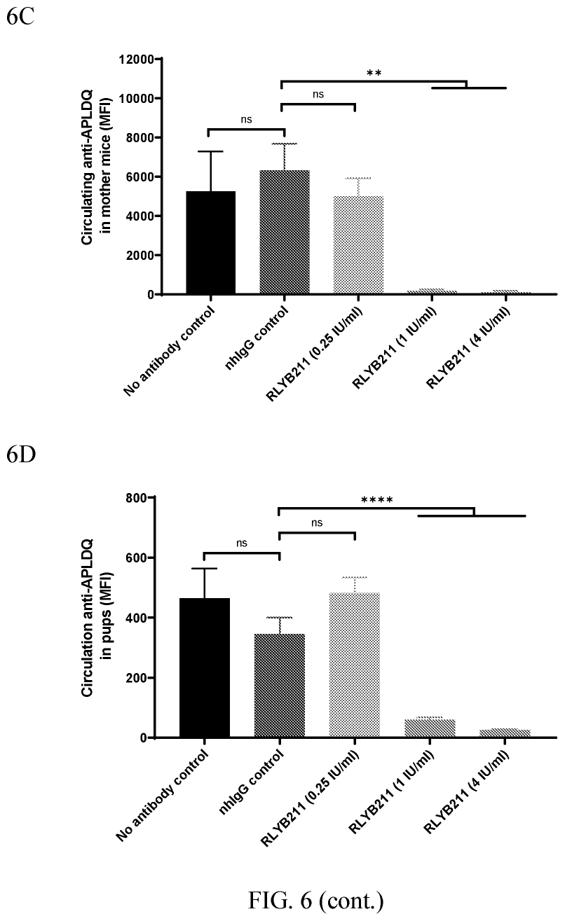

To assess the ability of prophylactic anti-HPA-1a antibody treatment to sustain prevention of alloimmunization to repeated immune challenges, a second round of prophylaxis with RLYB211 and APLDQ platelet transfusion was performed 21 days after the initial challenge. Again, doses projected to achieve 1-4 IU/ml of RLYB211 significantly reduced or completely prevented APLDQ alloimmunization (FIG. 6A).

Female mice that had been exposed to APLDQ-positive murine platelets in the presence of RLYB211, when bred with APLDQ-positive males, gave birth to pups with platelet counts that were elevated in direct proportion to the dose of the antibody that had been given to the dam, with little protection at ˜0.25 IU/ml, and nearly full protection at ˜4 IU/ml (FIG. 6B). Anti-APLDQ maternal alloantibody titers in the dams (FIG. 6C) and neonates (FIG. 6D) showed similar dose-dependent reductions that corresponded inversely to neonatal platelet counts.

### Example 5. Prevention of Alloimmunization by Anti-HPA-1a Antibodies

Pregnant women at risk for FNAIT are administered a prophylactic regimen of anti-HPA-1a antibodies. The prophylactic regimen maintains the highest safe exposure of anti-HPA-1a antibodies throughout the entire second and third trimesters and immediately following parturition. Based on an anti-HPA-1a value of 3 IU/mL, identified as the threshold for the occurrence of anti-HPA-1a antibody mediated fetal/neonatal thrombocytopenia (Killie et al., 2008; Bertrand et al., 2006), a clinical pharmacology model has been developed to maintain the exposure of therapeutic anti-HPA-1a antibodies at or below a target threshold (Cₘₐₓ) that is about 5-10-fold below the threshold value for development of neonatal thrombocytopenia.

Subjects are administered, via subcutaneous injection, an initial dose of anti-HPA1a antibodies, followed by a weekly maintenance dose to rapidly eliminate any HPA-1a positive fetal platelets from maternal circulation. Administration is initiated preferably between gestational weeks 10 and 14, and is repeated weekly until parturition. A final dose is administered within about 72 hours after parturition.

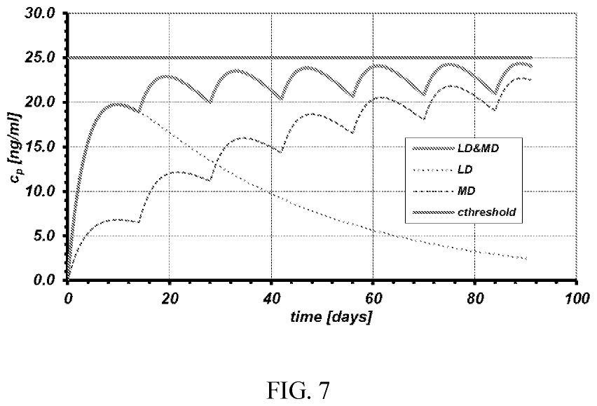

The administration regimen is designed to maximize the capacity of the anti-HPA-1a antibody to neutralize fetal antigen over the course of treatment by limiting fluctuations in plasma concentration from peak to trough. Pharmacokinetics simulations were performed to predict systemic exposures for one exemplary dosing regimen of the invention, using a loading/induction dose of 0.29 mg monoclonal anti-HPA-1a antibody, followed two weeks later by biweekly maintenance doses of 0.1 mg monoclonal anti-HPA-1a antibody, compared with a single dose of 0.29 mg monoclonal anti-HPA-1a antibody (FIG. 7). The initial dose achieves an initial peak plasma concentration after about 9.8 days. Subsequent peak plasma concentrations increase somewhat over each biweekly maintenance dose, reaching their overall maximum about 5 days after the final maintenance dose.

SEQUENCES

SEQ ID NO: 1-RLYB212 Heavy Chain Variable (V_{H}) Region
Gln Val Gln Leu Gln Gln Ser Gly Pro Gly Leu Val Lys Pro Ser Gln Thr Leu Ser Leu Thr Cys
Ala Ile Ser Gly Asp Ser Val Ser Ser Asn Ser Ala Ala Trp Asn Trp Ile Arg Gln Ser Pro Ser Arg
Gly Leu Glu Trp Leu Gly Arg Thr Tyr Phe Arg Ser Asn Trp Tyr Asn Asp Tyr Ala Ala Ser Val
Lys Ser Arg Ile Thr Ile Asn Gln Asp Thr Ser Lys Asn Gln Leu Ser Leu Gln Leu Asn Ser Val Thr
Pro Glu Asp Thr Ala Val Tyr Tyr Cys Ala Arg Asp Gly Ala Trp Gly Gly Ser Ser Trp Trp Pro
Gly Leu Pro His His Tyr Tyr Ser Gly Met Asp Val Trp Gly Gln Gly Thr Thr Val Thr Val Ser Ser
SEQ ID NO: 2-RLYB212 Light Chain Variable (V_{L}) Region
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg Leu
Leu Ile Tyr Asp Ala Ser Lys Arg Ala Thr Gly Ile Pro Ala Arg Phe Ser Gly Ser Gly Ser Gly Thr
Asp Phe Ser Leu Thr Ile Arg Ser Leu Glu Pro Glu Asp Phe Ala Val Tyr Tyr Cys Gln Gln Arg
Ser Asp Trp Gln Gly Leu Thr Phe Gly Gly Gly Thr Lys Val Glu Ile Lys
SEQ ID NO: 3-RLYB212 V_{H} Complementarity Determining Region (CDR) 1
Gly Asp Ser Val Ser Ser Asn Ser Ala Ala
SEQ ID NO: 4-RLYB212 V_{H} CDR 2
Thr Tyr Phe Arg Ser Asn Trp Tyr Asn
SEQ ID NO: 5-RLYB212 V_{H} CDR 3
Ala Arg Asp Gly Ala Trp Gly Gly Ser Ser Trp Trp Pro Gly Leu Pro His His Tyr Tyr Ser Gly Met
Asp Val
SEQ ID NO: 6-RLYB212 V_{L} CDR 1
Gln Ser Val Ser Ser Tyr
SEQ ID NO: 7-RLYB212 V_{L} CDR 2
Asp Ala Ser
SEQ ID NO: 8-RLYB212 V_{L} CDR 3
Gln Gln Arg Ser Asp Trp Gln Gly Leu Thr
SEQ ID NO: 9-RLYB212 Heavy Chain
Gln Val Gln Leu Gln Gln Ser Gly Pro Gly Leu Val Lys Pro Ser Gln Thr Leu Ser Leu Thr Cys
Ala Ile Ser Gly Asp Ser Val Ser Ser Asn Ser Ala Ala Trp Asn Trp Ile Arg Gln Ser Pro Ser Arg
Gly Leu Glu Trp Leu Gly Arg Thr Tyr Phe Arg Ser Asn Trp Tyr Asn Asp Tyr Ala Ala Ser Val
Lys Ser Arg Ile Thr Ile Asn Gln Asp Thr Ser Lys Asn Gln Leu Ser Leu Gln Leu Asn Ser Val Thr
Pro Glu Asp Thr Ala Val Tyr Tyr Cys Ala Arg Asp Gly Ala Trp Gly Gly Ser Ser Trp Trp Pro
Gly Leu Pro His His Tyr Tyr Ser Gly Met Asp Val Trp Gly Gln Gly Thr Thr Val Thr Val Ser Ser
Ala Ser Thr Lys Gly Pro Ser Val Phe Pro Leu Ala Pro Ser Ser Lys Ser Thr Ser Gly Gly Thr Ala
Ala Leu Gly Cys Leu Val Lys Asp Tyr Phe Pro Glu Pro Val Thr Val Ser Trp Asn Ser Gly Ala
Leu Thr Ser Gly Val His Thr Phe Pro Ala Val Leu Gln Ser Ser Gly Leu Tyr Ser Leu Ser Ser Val
Val Thr Val Pro Ser Ser Ser Leu Gly Thr Gln Thr Tyr Ile Cys Asn Val Asn His Lys Pro Ser Asn
Thr Lys Val Asp Lys Lys Val Glu Pro Lys Ser Cys Asp Lys Thr His Thr Cys Pro Pro Cys Pro
Ala Pro Glu Leu Leu Gly Gly Pro Ser Val Phe Leu Phe Pro Pro Lys Pro Lys Asp Thr Leu Met
Ile Ser Arg Thr Pro Glu Val Thr Cys Val Val Val Asp Val Ser His Glu Asp Pro Glu Val Lys Phe
Asn Trp Tyr Val Asp Gly Val Glu Val His Asn Ala Lys Thr Lys Pro Arg Glu Glu Gln Tyr Asn
Ser Thr Tyr Arg Val Val Ser Val Leu Thr Val Leu His Gln Asp Trp Leu Asn Gly Lys Glu Tyr
Lys Cys Lys Val Ser Asn Lys Ala Leu Pro Ala Pro Ile Glu Lys Thr Ile Ser Lys Ala Lys Gly Gln
Pro Arg Glu Pro Gln Val Tyr Thr Leu Pro Pro Ser Arg Asp Glu Leu Thr Lys Asn Gln Val Ser
Leu Thr Cys Leu Val Lys Gly Phe Tyr Pro Ser Asp Ile Ala Val Glu Trp Glu Ser Asn Gly Gln
Pro Glu Asn Asn Tyr Lys Thr Thr Pro Pro Val Leu Asp Ser Asp Gly Ser Phe Phe Leu Tyr Ser
Lys Leu Thr Val Asp Lys Ser Arg Trp Gln Gln Gly Asn Val Phe Ser Cys Ser Val Met His Glu
Ala Leu His Asn His Tyr Thr Gln Lys Ser Leu Ser Leu Ser Pro Gly Lys
SEQ ID NO: 10-RLYB212 Light Chain
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg Leu
Leu Ile Tyr Asp Ala Ser Lys Arg Ala Thr Gly Ile Pro Ala Arg Phe Ser Gly Ser Gly Ser Gly Thr
Asp Phe Ser Leu Thr Ile Arg Ser Leu Glu Pro Glu Asp Phe Ala Val Tyr Tyr Cys Gln Gln Arg
Ser Asp Trp Gln Gly Leu Thr Phe Gly Gly Gly Thr Lys Val Glu Ile Lys Arg Thr Val Ala Ala
Pro Ser Val Phe Ile Phe Pro Pro Ser Asp Glu Gln Leu Lys Ser Gly Thr Ala Ser Val Val Cys Leu
Leu Asn Asn Phe Tyr Pro Arg Glu Ala Lys Val Gln Trp Lys Val Asp Asn Ala Leu Gln Ser Gly
Asn Ser Gln Glu Ser Val Thr Glu Gln Asp Ser Lys Asp Ser Thr Tyr Ser Leu Ser Ser Thr Leu Thr
Leu Ser Lys Ala Asp Tyr Glu Lys His Lys Val Tyr Ala Cys Glu Val Thr His Gln Gly Leu Ser
Ser Pro Val Thr Lys Ser Phe Asn Arg Gly Glu Cys

### REFERENCES

ACOG Practice Bulletin No. 207: Thrombocytopenia in Pregnancy. Obstet Gynecol. 2019; 133(3):e181-e193. doi:10.1097/AOG.0000000000003100.
Allen D, et al. Collaborative study to establish the first international standard for quantitation of anti-HPA-1a. Vox Sang. 2005; 89: 100-104.
Baker E K, et al. A genetic analysis of integrin function: Glanzmann thrombasthenia in vitro. Proc Natl Acad Sci USA. 1997; 94(5):1973-1978.
Bertrand G, et al. Predictive value of sequential maternal anti-HPA-1a antibody concentrations for the severity of fetal alloimmune thrombocytopenia. J Thromb Haemost. 2006; 4(3):628-637.
Brojer E, et al. Fetal/Neonatal Alloimmune Thrombocytopenia: Pathogenesis, Diagnostics and Prevention. Arch Immunol Ther Exp (Warsz). 2016; 64(4):279-290.
Campbell K, et al. A modified rapid monoclonal antibody-specific immobilization of platelet antigen assay for the detection of human platelet antigen (HPA) antibodies: a multicentre evaluation. Vox Sanguinis 2007; 93:289-97.
Coller B S, et al. A murine monoclonal antibody that completely blocks the binding of fibrinogen to platelets produces a thrombasthenic-like state in normal platelets and binds to glycoproteins IIb and/or IIIa. J Clin Invest. 1983; 72(1):325-338.
Coller B S, et al. Abolition of in vivo platelet thrombus formation in primates with monoclonal antibodies to the platelet GPIIb/IIIa receptor. Correlation with bleeding time, platelet aggregation, and blockade of GPIIb/IIIa receptors. Circulation. 1989; 80:1766-1774.
Defilippi P, et al. Differential distribution and modulation of expression of alpha 1/beta 1 integrin on human endothelial cells. J Cell Biol. 1991; 114(4):855-863.
Eksteen M, et al. Characterization of a human platelet antigen-1a-specific monoclonal antibody derived from a B cell from a woman alloimmunized in pregnancy. J Immunol. 2015; 194(12):5751-5760.
Espinoza J P, et al. Fetal and neonatal alloimmune thrombocytopenia. Rev Obstet Gynecol. 2013; 6(1):e15-e21.
Göhner C, et al. Immune-modulatory effects of syncytiotrophoblast extracellular vesicles in pregnancy and preeclampsia. Placenta. 2017; 60 Suppl 1:S41S51.
Gold H K, et al. Pharmacodynamic study of F(ab′)2 fragments of murine monoclonal antibody 7E3 directed against human platelet glycoprotein IIb/IIIa in patients with unstable angina pectoris. J Clin Invest. 1990; 86:651-659.
Kiefel V, et al. Monoclonal antibody-specific immobilization of platelet antigens (MAIPA): a new tool for the identification of platelet-reactive antibodies. Blood 1987; 70:1722-26.
Killie M K, et al. A prospective study of maternal anti-HPA 1a antibody level as a potential predictor of alloimmune thrombocytopenia in the newborn. Haematologica. 2008; 93(6):870-877.
Kjeldsen-Kragh J, et al. Towards a prophylactic treatment of HPA-related foetal and neonatal alloimmune thrombocytopenia. Curr Opin Hematol. 2012; 19(6):469474.
Kohler, et al. Continuous cultures of fused cells secreting antibody of predefined specificity. Nature 1975; 256:495.
Lefranc M P et al., IMGT unique numbering for immunoglobulin and T cell receptor variable domains and Ig superfamily V-like domains. Dev Comp Immunol. 2003; 27:55-77.
Lieberman L, et al. Fetal and neonatal alloimmune thrombocytopenia: recommendations for evidence-based practice, an international approach. Br J Haematol. 2019; 185(3):549-562.
Newman P J, et al. The human platelet alloantigens, PlA1 and PlA2, are associated with a leucine33/proline33 amino acid polymorphism in membrane glycoprotein IIIa, and are distinguishable by DNA typing. J Clin Invest. 1989; 83(5):1778-1781.
Pacheco L D, et al. Fetal and neonatal alloimmune thrombocytopenia: a management algorithm based on risk stratification. Obstet Gynecol. 2011; 118(5):1157-1163.
Regan F, et al. Prenatal Management of Pregnancies at Risk of Fetal Neonatal Alloimmune Thrombocytopenia (FNAIT): Scientific Impact Paper No. 61. BJOG. 2019; 126(10):e173-e185.
Riches A C, et al. Blood volume determination in the mouse. J Physiol, 1973; 228(2):279-284.
Tiller H, et al. Toward a prophylaxis against fetal and neonatal alloimmune thrombocytopenia: induction of antibody-mediated immune suppression and prevention of severe clinical complications in a murine model. Transfusion. 2012; 52(7):1446-1457.
Vetlesen A, et al. Recovery, survival, and function of transfused platelets and detection of platelet engraftment after allogeneic stem cell transplantation. Transfusion 2012; 52:1321-1332.
Vitiello G, et al. Intravenous immunoglobulin therapy: a snapshot for the internist. Intern Emerg Med. 2019; 14(7):1041-1049.
WHO International Standard: Anti-HPA-1a Standard (100 IU). NIBSC code: 03/152. National Institute for Biological Standards and Control, Hertfordshire, United Kingdom.
Zhi H, et al. High-resolution mapping of the polyclonal immune response to the human platelet alloantigen HPA-1a (Pl(A1)). Blood Adv. 2018; 2(21):3001-3011.
Zhi H, et al. An Authentic Murine Model of Fetal/Neonatal Alloimmune Thrombocytopenia. Blood 2019; 134 (Suppl. 1):97.
Zhou Y, et al. Human cytotrophoblasts adopt a vascular phenotype as they differentiate. A strategy for successful endovascular invasion? J Clin Invest. 1997; 99(9):2139-2151.

The present invention is further described by the following claims.

1. A method of preventing alloimmunization with HPA-1a in a subject, wherein the subject is an HPA-1a-negative pregnant woman, the method comprising parenterally administering to the subject multiple doses of a pharmaceutical composition comprising an anti-HPA-1a antibody,
wherein the anti-HPA-1a antibody does not bind HPA-1b;
wherein an initial dose of the pharmaceutical composition is administered between gestational weeks 10 and 16 of pregnancy;
wherein maintenance doses of the pharmaceutical composition are administered after the initial dose, at regular dose intervals throughout pregnancy; and
wherein at least one dose is administered within 72 hours post-parturition.

2. A method of preventing fetal and neonatal alloimmune thrombocytopenia (FNAIT) caused by maternal alloimmunization with HPA-1a in a fetus of an HPA-1a-negative human subject, the method comprising parenterally administering to the subject multiple doses of a pharmaceutical composition comprising an effective amount of an anti-HPA-1a antibody,
wherein the anti-HPA-1a antibody does not bind HPA-1b;
wherein an initial dose of the pharmaceutical composition is administered between gestational weeks 10 and 16;
wherein maintenance doses of the pharmaceutical composition are administered after the initial dose, at a regular dose interval throughout pregnancy; and
wherein at least one dose is administered within 72 hours post-parturition.

3. The method of claim 1 wherein the anti-HPA-1a antibody is a polyclonal antibody.

4. The method of claim 3, wherein the pharmaceutical composition is anti-HPA-1a gamma globulin.

5. The method of claim 1, wherein the anti-HPA-1a antibody is a monoclonal antibody.

6. The method of claim 5, wherein the monoclonal antibody is RLYB212.

7. The method of claim 1, wherein the pharmaceutical composition is administered via subcutaneous injection.

8. The method of claim 1, wherein the pharmaceutical composition is administered via intravenous infusion.

9. The method of claim 1, wherein the pharmaceutical composition is self-administered.

10. The method of claim 1, wherein the pharmaceutical composition is administered from a vial and syringe, a pre-filled syringe, a pen injector, or an autoinjector.

11. The method of claim 1, wherein the regular dose interval is once weekly.

12. The method of claim 1, wherein the regular dose interval is twice weekly.

13. The method of claim 1, wherein the regular dose interval is once every two weeks.

14. The method of claim 1, wherein the initial dose is the same as the maintenance dose.

15. The method of claim 1, wherein the initial dose is higher than the maintenance dose.

16. The method of claim 7, wherein Tₘₐₓ of the anti-HPA-1a antibody is at the end of infusion.

17. The method of claim 8, wherein Tₘₐₓ of the initial dose of anti-HPA-1a antibody is 5 to 15 days after administration.

18. The method of claim 1, wherein C_{threshold} of the anti-HPA-1a antibody is about 8.5 ng/mL.

19. The method of claim 1, wherein the steady-state plasma concentration of the anti-HPA-1a antibody is about 6.5 ng/mL to about 8.5 ng/mL.

20. The method of claim 1, wherein the initial dose of the anti-HPA-1a antibody is 5-400 kg.

21. The method of claim 1, wherein the initial dose of the anti-HPA-1a antibody is 50-350 μg.

22. The method of claim 1, wherein the maintenance dose of the anti-HPA-1a antibody is 5-400 μg.

23. The method of claim 1, wherein the maintenance dose of the anti-HPA-1a antibody is 5-150 μg.

24. The method of claim 1, wherein the subject is HLA-DRB3*01:01 positive.

25. A pharmaceutical composition comprising an effective amount of an anti-HPA-1a antibody for use in a method of preventing alloimmunization with HPA-1a in a subject, wherein the subject is an HPA-1a-negative pregnant woman, the method comprising parenterally administering to the subject multiple doses of a pharmaceutical composition comprising an anti-HPA-1a antibody,
wherein an initial dose of the pharmaceutical composition is administered between gestational weeks 10 and 16;
wherein maintenance doses of the pharmaceutical composition are administered after the initial dose, at a regular dose interval throughout pregnancy; and
wherein at least one dose is administered within 72 hours post-parturition.

26. A pharmaceutical composition comprising an effective amount of an anti-HPA-1a antibody for use in a method of preventing fetal and neonatal alloimmune thrombocytopenia (FNAIT) caused by maternal alloimmunization with HPA-1a in a fetus of an HPA-1a-negative subject, the method comprising parenterally administering to the subject multiple doses of a pharmaceutical composition comprising an effective amount of an anti-HPA-1a antibody,
wherein an initial dose of the pharmaceutical composition is administered between gestational weeks 10 and 16;
wherein maintenance doses of the pharmaceutical composition are administered after the initial dose, at a regular dose interval throughout pregnancy; and
wherein at least one dose is administered within 72 hours post-parturition.
